SEQUENCE LISTING (6 sequences)

SEQ ID NO: 1 - DNA, 11 bp, artificial sequence
  synthetic sonstruct
auaccauaugc

SEQ ID NO: 2 - DNA, 11 bp, artificial sequence
  synthetic construct
uaugguauacg

SEQ ID NO: 3 - DNA, 15 bp, artificial sequence
  synthetic construct
aaaaauaccauaugc

SEQ ID NO: 4 - DNA, 15 bp, artificial sequence
  synthetic construct
aaaauaugguauacg

SEQ ID NO: 5 - DNA, 21 bp, artificial sequence
  sythetic construct
aaaagccguagguugcccaaa

SEQ ID NO: 6 - DNA, 61 bp, artificial sequence
  synthetic construct
gggcgacccugaugagucuggauaccaugcaugaugcaccuuggcagucuuacgaaacggu

(19) United States
(12) Patent Application Publication
(10) Pub. No.: US 2014/0271771 A1
(43) Pub. Date: Sep. 18, 2014

(54) IMPLANTABLE OBJECT COMPRISING SELECTIVELY DEGRADABLE COPOLYMERS FOR IMPROVED EXPLANTABILITY

(71) Applicant: Biotronik AG, Bülach (CH)

(72) Inventors: Jens Ulmer, Kuesnacht (CH); Michael Diebold, Berlin (DE)

(73) Assignee: Biotronik AG, Bülach (CH)

(21) Appl. No.: 14/196,522

(22) Filed: Mar. 4, 2014

Related U.S. Application Data
(60) Provisional application No. 61/781,023, filed on Mar. 14, 2013.

(51) Int. Cl. A61L 31/10; A61L 31/14

(52) U.S. Cl. CPC A61L 31/10; A61L 31/148
USPC 424/423; 523/113; 623/1.46; 607/119; 606/108; 606/129

(57) ABSTRACT

Implantable object, comprising a layer, comprising a copolymer that contains an aptamer, wherein bonds in the copolymer can be broken in accordance with a binding event at the aptamer, such that the copolymer degrades.

### CROSS REFERENCE TO RELATED APPLICATIONS

This application claims benefit of priority to U.S. provisional patent application Ser. No. 61/781,023 filed Mar. 14, 2013; the entire content of which is herein incorporated by reference in its entirety.

### TECHNICAL FIELD

The invention relates to an implantable object, comprising a layer, comprising a copolymer with an aptamer sub-unit, wherein bonds in the copolymer can be broken in accordance with a binding event at the aptamer such that the copolymer degrades. This enables an improved explantability. In particular, the invention relates to implantable objects in which the copolymers are adapted such that they are degraded in the implanted state with the occurrence of a predetermined stimulus, which may be produced naturally in the body or may be foreign to the body.

### BACKGROUND

The degradation of implants or parts of implants, in this case in particular of coatings, is a process to which much attention is devoted for medical reasons. On the one hand, it is important to check the rate of decomposition of implants in the body in order to ensure that the implants retain their function for a sufficiently long period of time. In the extreme case, degradation is thus completely undesired. On the other hand, a large number of materials in the body leads to incompatibility reactions. This can be counteracted by covering the implants with coatings. Coatings that positively influence the ingrowth behavior and therefore the acceptance of the implant in the body since they are subject to controlled decomposition by the natural environment of the body, are known in the prior art.

It may also be desirable for implants to be completely decomposed in the body, for example because a stent is no longer required after a sufficient period of mechanical support of the respective vessel due to the bodily reactions.

A further approach with which it is desirable for parts of implants to be degraded is as follows: the outer layers of an implant are charged with active ingredients, which primarily are not released by diffusion, but by the degradation of the layer carrying the active ingredient. A delayed release of the active ingredient is therefore possible. On this basis, it is beneficial to control the degradation behavior of implants or parts of implants as precisely as possible.

Polymers exist which, under physiological conditions, have constant decomposition behavior. For example, polyesters, polyorthoesters, polyalcohols, polyethers, polyamides, polyurethanes and polyanhydrides demonstrate a constant degradation rate under the influence of enzymes (such as esterases, lipases, dehydrogenases or oxygenases). Furthermore, most of these polymers are subject to general hydrolysis under physiological conditions and are thus decomposed after contact with water over a relatively long period of time. The degradation rate of these polymers can be controlled by their molecular composition (use of copolymers, polymer mixtures, type and degree of crosslinking) by combining the proportions of hydrolysis-sensitive binding units with non-degradable units or by varying hydrophilic and hydrophobic proportions. Constant degradation rates from a few days to a number of months can thus be implemented.

However, constant degradation behavior is an insufficient solution in many cases: It is desirable for the degradation in particular of polymer coatings on implants to be controlled in a manner that is as sophisticated as possible. This applies in particular for the situation in which an implant is to be removed again from the body: In particular in the case of implants that are left in the body for a relatively long period of time, ingrowth processes regularly occur. This is generally desired, since stability of the implant in the body is thus ensured. At the latest at the moment in time at which the ingrowth process is complete, it is therefore desirable for an implant to have a stable surface.

In many cases, it is desirable however or even necessary to remove an implant again from the body. For example, the reason for this may be that the implant has performed its function and is not required further, or that the implant is damaged and has to be replaced. A typical example for the latter case is an electron break in heart pacemakers. Due to the ingrowth of the implant into the body, which is desired per se, explantation is considerably impaired however. Considerable tissue damage is often caused during explantation due to the fixed contact between the natural tissue of the body and the implant surface. It is accordingly desirable for a coating of an implant to then only degrade when the implant is to be removed again from the body. To this end, polymers exist in the prior art which modify the decomposition behavior as a result of selective external influence. These influences include, inter alia, changes of pH, ion strength, and temperature. Even irradiation with electromagnetic energy and other ionizing beams or the effect of electric or magnetic fields may likewise be used to trigger degradation.

For example, polymers from the group of acrylamide, methacrylamide, dimethylaminoethyl methacrylate or a derivative of acrylamide, methacrylamide, dimethylaminoethyl methacrylate or poly n-isopropylacrylamide and poly-n-isopropylacrylamide-co-allylamine and preferably PNIPAM with poly(p-dioxanone) as a hard segment in combination, for example, with poly-L-lactide as a structure-giving and degradable element thus have a temperature-dependant swelling capacity in water that falls by 30-50% with a temperature increase of 10 K. These variable swelling states then make it possible to control a hydrolytic decomposition of ester functions due to the temperature-dependant water content.

The previously known solutions have a series of disadvantages however:

The influence of the degradation, based on pH, ion strength and/or temperature change, can only be carried out in vivo with great difficulty since the human organism reacts very sensitively to change of these parameters and only tolerates a very small deviation from the normal state. By contrast, electromagnetic radiation can only penetrate insufficiently in low energy ranges, whereby polymers in deeper tissue layers (for example on stents in coronary vessels) cannot be reached. By contrast, high-energy radiation constitutes a fundamental potential danger for the organism, but could penetrate cellular tissue very deeply (X-rays). Electric and magnetic fields do not demonstrate the above-mentioned restrictions, but require a significant outlay in terms of equipment, which considerably impairs the use of these methods in the medical field. In addition, a prompt, automatic reaction of the system is impaired thereby.

### SUMMARY

The object of the invention is therefore to specify an implantable object, which is improved for explantation in terms of the controllability of its degradation behavior.

This object is achieved by an implantable object, comprising a solid body (implant main body) and a layer arranged thereon, comprising a copolymer that contains an aptamer, wherein bonds in the copolymer can be broken in accordance with a binding event at the aptamer such that the copolymer degrades.

Here, an implantable object within the meaning of the invention is an object that is designed such that it can be introduced into the human or animal body and can remain therein for a period of at least 5 days.

Here, implantable objects within the meaning of the present invention are such objects that are introduced into the body in order to perform functions that are not ensured by the aptamer-containing layer alone. For example, a function of an accordingly coated stent is thus primarily a supporting function for a vessel, whereas the function of the aptamer-containing layer is to ensure improved explantability.

For the implantable objects according to the invention, it is accordingly important to understand that the aptamer-containing layers within the meaning of the present invention still surround or at least partly cover a material deviating from said layers. This material is, or comprises, at least one solid material, which is referred to by the term “solid body”. Here, this material is generally the main component of the implant (for example the corresponding alloy in the case of a stent).

An aptamer within the meaning of this invention is a polymer formed of ribonucleotides and/or deoxyribonucleotides as well as analogues thereof, such as nucleotide analogues with peptide components (PNAs), nucleotides with phosphorothioates, nucleotide analogues with morpholino components and LNAs (locked nucleic acids). Here, an additional chemical bond is introduced between the 2′O atom and the 4′C atom of the ribose group within the RNA base. This makes it possible to fix the ribose in the 3′-endo conformation, which significantly increases the stability of said ribose compared to endonucleases and exonucleases.

Within the meaning of the present text, “aptamers” demonstrate the property of binding to organic molecules with conformational change. This affinity can be massively increased for example by successive selections of different chain molecules consisting of the above-mentioned components (SELEX process).

A preferred variant of an aptamer is a Spiegelmer. In this case, the aptamer is formed from non-naturally occurring nucleotides to provide nucleotides enantiomeric to the naturally occurring nucleotides, at least in some regions. For example, the replacement of the natural D-ribose by the artificial L-ribose in the sugar unit of the nucleotide is to be mentioned for example.

Within the meaning of this invention, the size of aptamers is preferably 10-100 and particularly preferably 10-50 nucleotides or analogues thereof.

A copolymer within the meaning of this invention is a polymer that, besides the aptamer, also comprises further repetitive monomer sub-units. A block copolymer is preferable within the meaning of the invention.

Within the meaning of the invention, “bonds in the copolymer” are covalent bonds, hydrogen bridge bonds and ionic interactions. This means that, within the meaning of the invention, a copolymer can be formed from various (block) sub-units, which are connected to one another by hydrogen bridge bonds. For example, this is the case if the copolymer sub-units each consist of an aptamer or a polyoligonucleotide complementary to this aptamer and a further polymer. These sub-units can thus be bonded together to form the copolymer via the hydrogen bridge bonds between the aptamer and the complementary strand formed in each case from a copolymer sub-unit.

Within the meaning of the present text, “degradation of the copolymer” means that the strength of the bonding of the overall polymer is weakened, which generally occurs as a result of bond breaking.

A binding event within the meaning of this invention is the attachment of at least one compound to a second compound or to a domain of the second compound. In the case of such a binding event, the domain of the second compound is preferably an aptamer. Here, the attachment may occur by any types of interactions, but ionic interactions, hydrogen bridge bonds and covalent bonds are preferred.

Within the meaning of the present invention, a layer that comprises an implantable object according to the invention is a three-dimensional structure in particular, which has two main faces (an inner face and an outer face, which are the two largest faces of the structure). Here, the root of the content of the outer or inner face is in each case greater than the average thickness of the space between these two faces by a factor greater than or equal to ≧10, preferably greater than or equal to ≧20, particularly preferably greater than or equal to ≧100. A layer within the meaning of the present text can be closed, which means that it completely surrounds a (three-dimensional) part of the implantable object, or can be open such that a completely closed three-dimensional part of the implant is not delimited by the layer. Within the meaning of the present invention, the layer is preferably closed. Furthermore, a layer can in turn be coated again, either in part or completely. This is also preferred in some cases. In most cases however, it is preferable for an aptamer-comprising layer to form at least part of the surface of the implantable object, preferably the entire surface of the implantable object.

Here, preferred layer thicknesses of the aptamer-containing layer of the object according to the invention are 0.1 μm-100 μm, but preferably 0.5 μm-10 μm, and particularly preferably between 1 μm and 5 μm.

In some cases it may be preferable for the aptamer-containing layer to comprise no physiologically active substances.

The advantage of an implantable object according to the invention lies in the fact that the stimulus that is to trigger the degradation of the copolymer can be selectively determined by means of a suitable selection of an aptamer. In particular, it is possible to set a stimulus in good time before explantation of the implantable object, said stimulus causing degradation of the aptamer-containing layer, As a result of this degradation, the connection between the tissue surrounding the implant and the implant itself would be weakened and ideally broken completely. The implant can thus be removed again without great difficulty, in particular without significantly affecting the natural tissue of the body.

An implantable object, wherein the object is adapted such that the binding event occurs in the state implanted in a human or animal body, triggered by a systemically supplied compound and/or with involvement of such a compound, is accordingly preferred within the meaning of the invention.

In this context, a systemically added compound is a compound that is added selectively to the organism in which the implantable object is implanted. Here, the systemically supplied compound is preferably a substance foreign to the body or a substance occurring naturally within the body, which is added to the body in a concentration such that a supraphysiological concentration is set in the body.

Here, preferred compounds to be added systemically are non-natural analogues of substances occurring naturally in the body. In this context and in many cases, substances that are used for diagnostic purposes and are accordingly compatible with the body, even in relatively large amounts, are particularly suitable and have also already provided the best results under test conditions. Three substance classes can be cited particularly preferably in this context, as follows:

Substance Class 1 (Halogenated Monosaccharides and Oligosaccharides and Sugar Alcohols):

Here, the OH group at the C2 or C3 atom of the sugar molecule is generally replaced by a halogen atom (F, Cl, Br).

### Preferred Examples

to glucose: 2-fluoro-2-deoxy-D-glucose (PET imaging contrast agent)
fructose: 2-fluoro-2-deoxy-D-fructose
galactose: 2-fluoro-2-deoxy-D-galactose
fluorinated sucrose, lactose, xylitol, sorbitol, maltite and/or erythrite are also preferred.

### Substance Class 2 (L-Monosaccharides and L-Oligosaccharides):

These are sugars that do not occur in nature and therefore in the food chain. Since these are chemically identical structures having physically similar properties, these substances can be taken without difficulty, even in relatively large amounts. The chiral differences between the individual monosaccharides can be identified by aptamers.

### Preferred Examples

L-pentoses and L-hexoses, in particular L-glucose, L-fructose, L-ribose, L-galactose, L-mannose, L-arabinose, L-xylose or sugar acids and amino sugars, in particular L-glucuronic acid, L-galacturonic acid, L-glucosamine.

### Substance Class 3 Halogenated Essential and Non-Essential Amino Acids:

### Preferred Examples

### 4-fluorophenylalanine and fluoroethyltyrosine

With methods known from the prior art, it is possible here for a person skilled in the art to develop aptamers that can perform the function desired for the invention (the breaking of bonds in the copolymer after reaction with the respective stimulus).

Here, in the meaning of this text, physiological conditions (and also physiological concentration) are the conventional physiological concentrations of substances that become established in the animal or preferably human organism with species-appropriate nutrition. However, this term preferably does not include any metabolic states to be defined as illness and in particular merely concentrations of the above-listed substance classes 1-3 of ≧1,500 ppb, preferably 1-1,200 ppb, more preferably 120-1,000 ppb, in each case based on the total weight of a, or the, bodily fluid contacting the implantable object.

The wording that “the binding event is triggered by a systemically supplied compound” means that the compound in question does not have to be involved directly in the binding event, but merely has to cause a reaction cascade that leads to the binding event.

“The binding event occurs with involvement of a compound” means that the compound is involved directly in the binding event.

### DESCRIPTION OF THE DRAWINGS

FIG. 1 shows a schematic view of a binding situation between a polymer unit and an aptamer according to the invention

FIG. 2 shows a schematic view of a binding situation between a polymer unit and an aptamer according to the invention

### DETAILED DESCRIPTION

The advantage that the binding event is triggered by the above-listed triggers lies in the fact that the conditions under which the copolymer degrades or the physiological situation entered when the copolymer degrades can thus be determined accurately.

An implantable object, wherein the aptamer, by binding to a strand at least partly complementary thereto, is involved in a crosslinking of individual copolymer (sub-)units, and the bond to the at least partly complementary strand is broken by the binding event, is preferred within the meaning of the invention.

If an affine sequence is known as an aptamer, this sequence can be combined in a manner suitable for a person skilled in the art with a polymer to form a copolymer. In addition, a sequence at least partly complementary to the aptamer can likewise be copolymerized with the same polymer. Copolymer sub-units, which can interconnect via the aptamer and a sequence at least partly complementary thereto to form the copolymer, are thus produced.

FIG. 1 shows a schematic view of a such a binding situation, wherein the reference signs mean as follows:
1 polymer unit
2 aptamer
3 strand complementary to the aptamer
4 molecule triggering a binding event (theophylline)
5 unbonded complementary sequence
6 aptamer binding to theophylline

An example for the situation described in FIG. 1 is the aptamer with the sequence AUACCAUAUGC (SEQ ID NO. 1), which has a greater affinity to theophylline than to its complementary sequence UAUGGUAUACG (SEQ ID NO. 2). If the aptamer and its complementary sequence are each formed as a side-chain function, for example introduced into linear acrylate polymers, a copolymer matrix can thus be formed, which degrades selectively with addition of theophylline.

By variation of the polymer blocks in the copolymer, the conditions can accordingly be selectively adjusted such that the degradation is triggered exclusively by addition of theophylline.

An implantable object according to the invention, wherein the aptamer interacts with a ribozyme or is part of a ribozyme and the activity of the ribozyme is shifted toward increased cleaving of bonds in the copolymer due to the binding event, is preferred.

In accordance with the invention, it is also possible for the aptamer to be component of a ribozyme as an alternative or an additional variant to the direct bond breaking of aptamers to form their complementary sequences.

Within the meaning of this text, a ribozyme is an RNA molecule with a precisely defined tertiary structure, which allows it to catalyze a specific chemical reaction. Here, the chemical reaction may preferably be accelerated many times over by the presence of a systemically supplied stimulus (allosteric interaction). The chemical reaction within the meaning of this text is preferably the hydrolysis reaction of a phosphorodiester bond of a second RNA molecule. In this case, this second RNA molecule constitutes part of the copolymer basic structure.

Ribozymes can be formed such that their enzyme activity relates to the cleaving of bonds within the copolymer. For example, ribozymes are thus able to cleave complementary RNA strands bonded to the ribozyme. Here, the system can be formed such that bonds of the molecular stimulus to the aptamer unit increase the enzyme activity of the ribozyme-bonded unit (preferably many times over). The complementary strand bonded to the ribozyme is thus cleaved and broken.

FIG. 2 illustrates such a situation. Here, the reference signs mean as follows:
1 polymer
2 aptamer
4 molecule triggering an event
8 ribozyme after binding event
12 ribozyme
14 stand break

With this preferred system, there is a further possibility for designing an implant according to the invention and using it for a series of applications.

One possible embodiment for variation with ribozymes is that an allosterically activatable ribozyme is used. Here, allosterically means that the binding event occurs at a point of the ribozyme other than the catalytically active center.

A specific embodiment of this version is the enzymatic active RNA sequence (what is known as a hammerhead ribozyme), which can cleave an RNA sequence with the sequence motif UAG (what is known as a IVup motif), once a binding event has occurred at the aptamer connected allosterically to the ribozyme (chemical stimulation) by means of a systemically supplied compound within the meaning of this invention. As a result of this binding event, the catalytic activity of the ribozyme increases many times over and thus makes it possible to selectively cleave the copolymer specifically at the UAG sequence motif by adding a chemical stimulus (see also R. Breaker, Engineered allosteric ribozymes as biosensor components).

An implantable object, wherein the copolymer is charged with an active ingredient, which can be released in an intensified manner in the event of degradation of the copolymer, is preferred. The charging of polymers and copolymers with active ingredients is known in the art and is described in detail for example in DE 10 2008 040 786 and is herein incorporated by reference in its entirety.

An object according to the invention, wherein the copolymer forms an outer protective layer for materials that are degradable under physiological conditions, is preferable for many applications.

This approach for example makes it possible to protect implants that are degradable constantly over time, such as AMS' (absorbable metal stents; stents consisting of a magnesium alloy, which is decomposed under physiological conditions), against degradation until the outer protective layer made of copolymer is decomposed by the contribution of the substance (stimulus) triggering the binding event such that the underlying layers are made accessible to the decomposition possible under physiological conditions.

For example, it is thus possible to define the exact moment in time at which the decomposition of a biologically degradable implant (for example of a stent made of a biologically degradable material) is to start.

For many applications, an object according to the invention with which the explantantion is facilitated by the degradation of the copolymer triggered by the binding event is preferred.

If the copolymer to be used in accordance with the invention is formed as an outermost coating of an implant, for example of a pacemaker electrode, it is thus made easier to remove the implant again, for example after functional loss such as an electrode break. Due to the systemic contribution of a stimulus (of a corresponding compound) triggering the binding event, the implant can be easily detached from the surrounding tissue since the outer coating made of the copolymer dissolves and thus loosens the contact between the implant (for example electrode) and tissue. The removal (explantation) of the implant is thus facilitated considerably. Preferred implantable objects are objects selected from the group consisting of heart pacemakers, stents, pacemaker electrodes, stimulation electrodes, cerebral catheters, joint replacements, implants for osteosynthesis, dental implants and plastic surgery implants.

Within the meaning of the invention it is also preferable for the copolymer to be used for the object according to the invention to also comprise, besides the aptamer (or a sequence complementary to the aptamer), monomers selected from the group consisting of acrylamide, hydroxyethyl methacrylate, ethylene glycol methacrylates, glucosyl ethyl methacrylate, hydroxypropyl methacrylamides, n-isopropylacrylamide, vinylpyrrolidone, vinylalcohol, vinylacetate, vinylacetate caprolactone, hydroxybutyrate, lactic acid, lactic-co-glycolic acid, ethylene glycol, propylene glycol.

The actual core of the invention is the use of a layer comprising a copolymer that contains an aptamer, wherein the layer is formed as described further above, for improving the explantability of an implantable object.

The approach of assisting explantation by means of rather selective degradation of a layer via the function of aptamers is novel in this instance. Here, it is also not necessarily the case that the aptamer-containing layer in the object, which is not yet implanted, forms the outermost layer: It may be desirable for further layers to be applied to the aptamer-containing layer to be used in accordance with the invention. For example, these layers may likewise be degradable and thus positively influence the ingrowth behavior. Here, these additional layers may or may not carry further active ingredients. The same is of course also true for the aptamer-containing layer, which is ultimately intended to facilitate explantation.

If further layers are applied to the layer assisting explantation, these may thus be decomposed within the scope of the ingrowth process for example such that the aptamer-containing layer is then the layer that comes into contact with the ingrowing tissue. It is also possible in principle however for the aptamer-containing layer to be located in an inner region of the implant when explantation is to be initiated. In this case too, it can perform its function in accordance with the invention provided it is merely ensured that the trigger for the degradation can reach the layer.

The invention also relates to a method for explanting an implant, said method comprising the following steps:
a) providing an implanted object as defined above as an implantable object, and
b) causing a binding event at the aptamer such that bonds are broken in the copolymer, whereby the copolymer degrades.

By means of this method, it is possible to ensure particularly easy explantation of implantable objects. Here, explantation in the broadest sense can also mean that the implantable object dissolves, that is to say it is not absolutely necessary to remove the implant by a purposeful (surgical) intervention. Ultimately, the dissolution of the aptamer-containing layer or the partial degradation results in the fact that the contact between the implant and the bodily tissue is reduced, and is preferably brought to zero. Such a bringing to zero is of course also then provided if the aptamer-containing layer, before degradation thereof, functioned as a protective layer of an implant that can be decomposed by the body. In this case, the explantation is simply a decomposition of the implant by the body.

### EXAMPLES

### Example 1

An example for producing an aptamer-containing copolymer will be described hereinafter:

### Production of Functional Aptamer Sensitive to Theophylline:

To couple the aptamers to a polymer, a methacryl group is introduced at the 5′ end thereof is (Acrydite, Mosaic, Technologies, Boston, Mass.). This enables the copolymerization of the RNA sequence, for example with acrylamide, hydroxyethyl methacrylate:

(SEQ ID NO. 3)
sequence 1: 5′acrydite-AAAA AUACCAUAUGC
(SEQ ID NO. 4)
sequence 2: 5′acrydite-AAAAUAUGGUAUACG

### Copolymer Formation:

Sequences 1 and 2 were each dissolved in an amount of 3 mM in 10 mM Tris (pH 8.0), 200 mM NaCl and 4% acrylamide (v/v) and degassed (solution A). A degassed mixture formed of 0.5 ml H₂O, 0.05 ammonium persulfate and 25 ml tetraethylmethylenediamine was then produced (solution B). 1.4% (v/v) of solution B were added to solution A and polymerized for 2 min

### Implant Coating Production

The implant main body [for example AMS (stent) or pacemaker electrode] is sprayed with methyl-2-cyanoacrylate for activation with exclusion of moisture. The activated implant main body is then sprayed with the partly polymerized solution and polymerized out for a further 10 min.

### Example 2

### Production of Functional Ribozyme Sensitive to Theophylline:

To couple the ribozyme to a polymer, a methacryl group is introduced to one of the sequences at the 5′ and 3′ end thereof (Acrydite, Mosaic Technologies, Boston, Mass.). This enables the copolymerization of the RNA sequence, for example with acrylamide hydroxyethyl methacrylate:

sequence 1:
(SEQ ID NO. 5)
5′acrydite-AAAAGCCGUAGGUUGCCCAAA-acrydite 3′
sequence 2:
(SEQ ID NO. 6)
5′pppGGGCGACCCUGAUGAGUCUGGAUACCAUGCAUGAUGCACCUUGGC
AGUCUUACGAAACGGU 3′

### Copolymer Formation:

Sequences 1 and 2 were each dissolved in an amount of 3 mM in 10 mM Tris (pH 8.0), 200 mM NaCl and 4% acrylamide (v/v) and degassed (solution A). A degassed mixture formed of 0.5 ml H₂O, 0.05 ammonium persulfate and 25 ml tetraethylmethylenediamine was then produced (solution B). 1.4% (v/v) of solution B were added to solution A and polymerized for 2 min.

### Implant Coating Production

The implant main body is sprayed with methyl-2-cyanoacrylate for activation with exclusion of moisture. The activated implant main body is then sprayed with the partly polymerized solution and polymerized out for a further 10 min.

### Preparation for Explantation

If the explantation of an implant, here a pacemaker electrode in particular, is necessary, the patient can thus be prepared for explantation within the meaning of the invention by supplying, for example intravenously, a molecule triggering a binding event, said molecule corresponding to the aptamer used to build up the implant coating. The supply of this molecule causes the polymer used to build up the implant coating to preferably be decomposed. To this end, the molecule triggering the binding event is supplied to the patient in a concentration that lies above the amount to be traced physiologically, but below the maximum allowed concentration.

In the case of explantation of a 40 cm long pacemaker electrode for example, which has been provided with a coating 10 μm thick, a total volume of 5*10⁻⁸ m³ therefore has to be decomposed. There is accordingly a total amount of 150 nmol aptamer within the coating. In order to decompose this completely, 150 nmol of the molecule triggering the binding event are also necessary. Since these are diluted accordingly in the blood, it may generally be expedient for the same concentration of the molecule triggering the binding event as is present in the implant coating to be provided in the blood. In the above-described case, that is to say 3 mM. In the case of glucose analogues as the molecule triggering the binding event, this molecule therefore lies in a concentration range corresponding to the physiologically relevant concentration of D-glucose. In many cases, it is preferable however for the concentration of the molecule triggering the binding event to be much lower, for example in the range of 1-1,500 ppb. A longer working time may then be necessary.

In the first proposed case, 50 ml for example of a 30 mM solution of 2-fluoro-deoxyglucose are thus infused in a physiological saline solution over a period of 10-20 min. After an incubation time of 2 h, the explantation preparations can be begun.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. An implantable object, comprising a solid body and, arranged thereon, a layer, comprising a copolymer that contains an aptamer, wherein bonds in the copolymer can be broken in accordance with a binding event at the aptamer, such that the copolymer degrades.

2. The implantable object as claimed in claim 1, wherein the object is adapted such that the binding event occurs in a state implanted in a human or animal body and is triggered by a systemically supplied compound and/or with involvement of such a compound.

3. The implantable object as claimed in claim 1, wherein the aptamer, by binding to a strand at least partly complementary thereto, is involved in a crosslinking of individual copolymer units, and the bond to the at least partly complementary strand is broken by the binding event.

4. The implantable object as claimed in claim 1, wherein the aptamer interacts with a ribozyme or is part of a ribozyme and the activity of the ribozyme is shifted toward increased cleaving of bonds in the copolymer as a result of the binding event.

5. The implantable object as claimed in claim 1, wherein the copolymer is charged with an active ingredient, which can be released in an intensified manner in the event of degradation of the copolymer.

6. The implantable object as claimed in claim 1, wherein the copolymer forms an outer protective layer for materials that are degradable under physiological conditions.

7. The implantable object as claimed in claim 1, wherein the binding event is triggered by a substance selected from the group consisting of a halogenated monosaccharide, a halogenated polyoligosaccharide, a halogenated sugar alcohol, L-monosaccharides, L-oligosaccharides and a halogenated amino acid.

8. The implantable object as claimed in claim 1, wherein the copolymer comprises monomers, selected from the group consisting of acrylamide, hydroxyethyl methacrylate, ethylene glycol methacrylates, glucosyl ethyl methacrylate, hydroxypropyl methacrylamides, n-isopropylacrylamide, vinylpyrrolidone, vinylalcohol, vinylacetate, vinylacetate caprolactone, hydroxybutyrate, lactic acid, lactic-co-glycolic acid, ethylene glycol, and propylene glycol.

9. The implantable object as claimed in claim 1, wherein the implantable object is selected from the group consisting of a heart pacemaker, a stent, pacemaker electrodes, stimulation electrodes, a cerebral catheter, a joint replacement, an implant for osteosynthesis, a dental implant and a plastic surgery implant.

10. Use of a layer, comprising a copolymer that contains an aptamer, wherein the layer is designed as described in claim 1, for improving the explantability of an implantable object.

11. A method for explanting an implant, the method comprising the following steps:
a) providing an implanted object as defined in claim 1 as an implantable object, and
b) causing a binding event at the aptamer such that bonds in the copolymer are broken, whereby the copolymer degrades.
